(19) United States
(12) Patent Application Publication
(10) Pub. No.: US 2025/0152384 A1
(43) Pub. Date: May 15, 2025

(54) VIRTUAL TACTILE IMPLEMENTATION DEVICE AND METHOD OF OPERATION THEREOF

(71) Applicant: Electronics and Telecommunications Research Institute, Daejeon (KR)

(72) Inventors: Kang-Ho PARK, Daejeon (KR); Yousung KANG, Daejeon (KR); Sung Q LEE, Daejeon (KR); Sung Jun JUNG, Seoul (KR); Young In CHOI, Hwaseong-si (KR); Hee Seong CHO, Seoul (KR)

(73) Assignee: Electronics and Telecommunications Research Institute, Daejeon (KR)

(21) Appl. No.: 18/935,939

(22) Filed: Nov. 4, 2024

(30) Foreign Application Priority Data
Nov. 9, 2023 (KR) 10-2023-0154779
May 21, 2024 (KR) 10-2024-0065928

(51) Int. Cl. A61F 2/58; A61F 2/50; A61N 1/36

(52) U.S. Cl. CPC A61F 2/586; A61N 1/36; A61F 2002/5059

(57) ABSTRACT

Disclosed is a virtual tactile implementation device, which includes one or more micro-displacement stimulation elements in close contact with a skin, a signal processor that applies a displacement signal to the micro-displacement stimulation element, and a power supply device that supplies electrical energy to the micro-displacement stimulation element and the signal processor, and the signal processor controls a waveform and an amplitude of the displacement signal to allow the micro-displacement stimulation element to selectively fire action potentials of a plurality of tactile receptors within the skin, and controls a time interval at which each of the action potentials is fired to allow the micro-displacement stimulation element to provide a sense of pressure or a sense of texture.

### CROSS-REFERENCE TO RELATED APPLICATIONS

This application claims priority under 35 U.S.C. § 119 to Korean Patent Application Nos. 10-2023-0154779 filed on Nov. 9, 2023, and 10-2024-0065928 filed on May 21, 2024, in the Korean Intellectual Property Office, the disclosures of which are incorporated by reference herein in their entireties.

### BACKGROUND

Embodiments of the present disclosure described herein relate to a virtual tactile implementation device, and more particularly, relate to a virtual tactile implementation device that selectively stimulates mechanoreceptors and a method of operating the same.

Recently, as interest in and demand for a Virtual Reality (VR), an Augmented Reality (AR), and a Mixed Reality (MR) increase, needs for implementing a realistic tactile sense, that is, a virtual tactile sense, such as touching an object through a skin, are increasing. It is relatively easy to implement a vibration sense using a vibration motor or a piezoelectric actuator, but a complex and bulky design is required to implement a pressure stimulation having a high-resolution, so it is uncomfortable to wear and expensive. A high-density electrode array may apply electrical stimulation to nearby tactile receptors, but the precision and/or resolution of a sense of pressure or a sense of texture is low.

A skin layer may be divided into an epidermis layer and a dermis layer depending on a depth. Looking at the distribution of the mechanoreceptors depending on the depth of the skin layer, Merkel cells exist in the lower layer of the epidermis, and Meissner's corpuscles exist in the dermis layer close to the epidermis layer. Pacinian corpuscles and Ruffini's endings are located deep in the dermis layer.

The functions of each of the mechanoreceptors are as follows. The Merkel cells in the epidermis layer have a very small receptive field size, so it is suitable for distinguishing the location of stimuli and has a slow stimulation response speed. The Meissner's corpuscles are located in the fingerprint area of palms and soles, and are sensitive to light touch. The Meissner's corpuscles have a narrow receptive field, and have a relatively fast stimulation response speed. The Pacinian corpuscles are densely distributed on fingers, are sensitive to fast and large vibrations, and have a wide receptive field and a very fast stimulation response speed. The Ruffini's endings are distributed at the tips of fingers, sense the degree of skin stretching, and have a wide receptive field and a slow stimulation response speed.

### SUMMARY

Embodiments of the present disclosure provide a virtual tactile implementation device that selectively stimulates mechanoreceptors and a method of operating the same.

According to the present disclosure, a device and a method for implementing virtual tactile senses such as high-resolution pressure senses and various textures are provided by selectively stimulating mechanoreceptors existing in a skin layer, using a micro-displacement stimulation element depending on waveform conditions of displacement stimulation to fire action potentials, and by controlling time intervals at which the action potentials of the mechanoreceptors are fired, using a signal processor.

According to an embodiment of the present disclosure, a virtual tactile implementation device includes one or more micro-displacement stimulation elements in close contact with a skin, a signal processor that applies a displacement signal to the micro-displacement stimulation element, and a power supply device that supplies electrical energy to the micro-displacement stimulation element and the signal processor, and the signal processor controls a waveform and an amplitude of the displacement signal to allow the micro-displacement stimulation element to selectively fire action potentials of a plurality of tactile receptors within the skin, and controls a time interval at which each of the action potentials is fired to allow the micro-displacement stimulation element to provide a sense of pressure or a sense of texture.

According to an embodiment of the present disclosure, a method of operating a virtual tactile implementation device including one or more micro-displacement stimulation elements which are in close contact with a skin and a signal processor, includes controlling, by the signal processor, a waveform and an amplitude of a displacement signal so as to be provided to the micro-displacement stimulation element, and providing, by the micro-displacement stimulation element, a tactile stimulation to the skin depending on the displacement signal, and the signal processor controls the waveform and the amplitude of the displacement signal to allow the micro-displacement stimulation element to selectively fire action potentials of a plurality of tactile receptors within the skin, and controls a time interval at which each of the action potentials is fired to allow the micro-displacement stimulation element to provide a sense of pressure or a sense of texture.

### BRIEF DESCRIPTION OF THE FIGURES

The above and other objects and features of the present disclosure will become apparent by describing in detail embodiments thereof with reference to the accompanying drawings.

FIG. 1 is a diagram illustrating a process of detecting and recognizing tactile stimuli.

FIG. 2 is a diagram illustrating a plurality of tactile receptors distributed on a skin.

FIG. 3 is a diagram illustrating action potentials that are fired when pressure stimuli or vibration stimuli are applied to a skin in a slowly adapting and a rapidly adapting.

FIG. 4 is a diagram illustrating a firing of action potentials of a plurality of tactile receptors when pressure stimuli are applied to a skin, divided by Merkel cells, Meissner's corpuscles, and Pacinian corpuscles.

FIG. 5 is a diagram illustrating a firing of action potentials of a plurality of tactile receptors when vibration stimuli are applied to a skin, divided by Merkel cells, Meissner's corpuscles, and Pacinian corpuscles.

FIG. 6 is a diagram illustrating a firing of action potentials of a plurality of tactile receptors when a specific texture stimulation is applied to a skin, divided by Merkel cells, Meissner's corpuscles, and Pacinian corpuscles.

FIG. 7 is a diagram illustrating a virtual tactile implementation device, according to some embodiments of the present disclosure.

FIG. 8 is a diagram illustrating a firing signal of an action potential of a Merkel cell when a displacement stimulation is applied to a skin, according to some embodiments of the present disclosure.

FIG. 9 is a diagram illustrating a firing signal of an action potential of a Merkel cell when a displacement stimulation with a series of sine waves having different pulse widths from the displacement stimulation of FIG. 8 is applied to a skin.

FIG. 10 is a diagram illustrating a firing rate curve representing a firing rate of an action potential of a Merkel cell over time when a pressure stimulation is applied to a skin.

FIG. 11 is a diagram illustrating artificially generating a firing rate curve graph of FIG. 10 to be similar to an experimental value, according to some embodiments of the present disclosure.

FIG. 12 is a diagram illustrating a firing rate curve graph generated by a method of FIG. 11.

FIG. 13 is a diagram illustrating a firing signal of an action potential of a Merkel cell over time, according to some embodiments of the present disclosure.

FIG. 14 is a graph illustrating conditions under which action potentials of Merkel cells, Meissner's corpuscles, and Pacinian corpuscles are fired depending on a frequency and an amplitude of a displacement signal so as to be similar to a sense of pressure when a displacement signal having a sine wave waveform is used in some embodiments of the present disclosure.

FIG. 15 is a diagram illustrating a firing of action potentials of a plurality of tactile receptors when a signal processor applies a displacement signal having a pulse signal waveform and changes an amplitude of a displacement signal and a time interval between pulses of the displacement signal, according to some embodiments of the present disclosure.

FIG. 16 is a diagram illustrating a firing of action potentials of a plurality of tactile receptors when a signal processor applies a displacement signal having a sine wave waveform and changes an amplitude and a pulse width of a displacement signal, according to some embodiments of the present disclosure.

FIG. 17 is a diagram illustrating a firing of action potentials of a plurality of tactile receptors when a texture stimulation is applied by varying a pulse width and an amplitude of a displacement signal when a displacement signal having a sine wave waveform is applied, according to some embodiments of the present disclosure.

FIG. 18 illustrates an example of a displacement stimulation of a micro-displacement stimulation element generated by varying an amplitude and a pulse width of the displacement stimulation, according to some embodiments of the present disclosure.

FIG. 19 is a diagram illustrating an enlarged view of an initial 0.3 second period of FIG. 18.

FIG. 20 is a side view and a front view of a contact device and a micro-displacement stimulation element, which are attached to a finger, according to some embodiments of the present disclosure.

FIG. 21 is a front view of a ring-shaped contact device having structure including a micro-displacement stimulation element inside, according to some embodiments of the present disclosure.

FIG. 22 is a diagram illustrating a contact device including a protrusion in a front view of FIG. 21.

FIG. 23 is a diagram illustrating a contact device, according to some embodiments of the present disclosure.

### DETAILED DESCRIPTION

Hereinafter, embodiments of the present disclosure will be described in detail and clearly to such an extent that an ordinary one in the art easily implements the present disclosure.

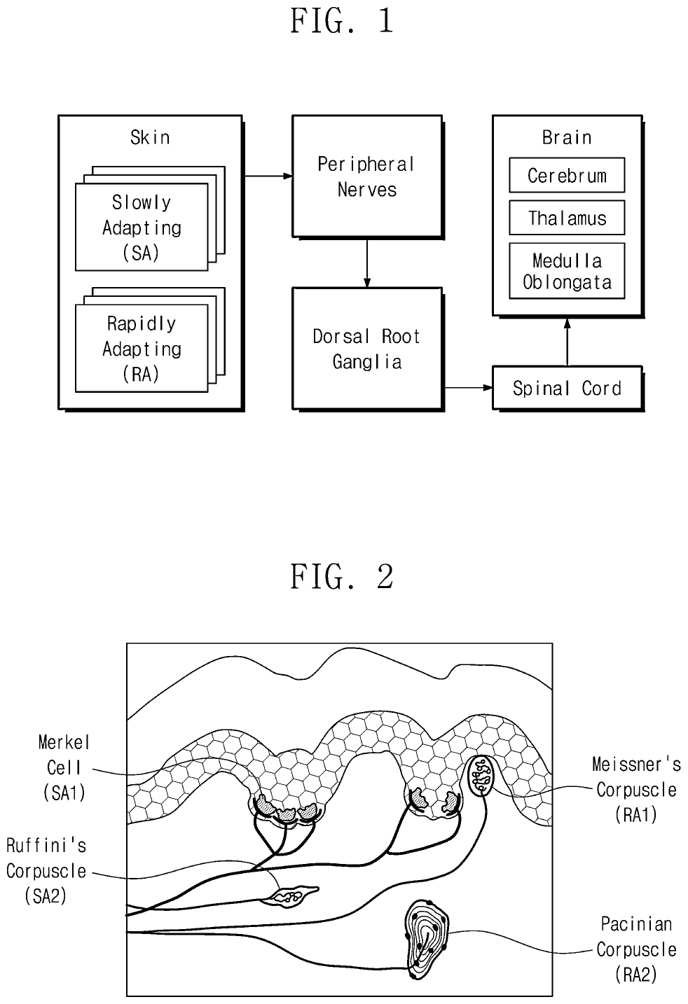

FIG. 1 is a diagram illustrating a process of detecting and recognizing tactile stimuli. When physical stimuli such as pressure, vibration, and texture are applied to a plurality of tactile receptors (e.g., mechanoreceptors) distributed on a skin such as fingers, action potentials may be fired from a plurality of tactile receptors and may be transferred to peripheral nerves.

The plurality of tactile receptors distributed on the skin may be broadly classified into a slowly adapting SA that responds slowly and a rapidly adapting RA that responds quickly. For example, the slowly adapting SA may include the Merkel cells that detect pressure or fine surface structures and the Ruffini corpuscles that detect skin elasticity and skin expansion. The rapidly adapting RA may include the Meissner's corpuscles that detect friction or slow and small vibrations and the Pacinian corpuscles that detect fast and strong vibrations over a relatively large area. Based on firing signals fired from these multiple tactile receptors, humans may sense fine and sophisticated touch (e.g., a sense of texture, a sense of pressure, etc.).

When mechanical stimuli such as various pressures, vibrations, and textures are detected by the tactile receptors distributed at the ends of the skin, firing signals of action potentials may occur at the ends of the tactile receptors depending on the type and the magnitude of the stimulation. The firing signals reach the peripheral nerves along the neurons, pass through a dorsal root ganglia, and are transferred along the spinal nerves of a spinal cord. The firing signals then reach a somatosensory area through the thalamus of the brain, and the cerebrum combines the firing signals to distinguish between a distribution and a magnitude of pressure, and a frequency of vibration, and a distribution and a magnitude, and from this, the pressure, the vibration, and even fine texture information may be recognized and distinguished.

FIG. 2 is a diagram illustrating a plurality of tactile receptors distributed on a skin. Referring to FIG. 2, a Merkel cell SA1 that mainly senses a sense of pressure as the slowly adapting, a Ruffini corpuscle SA2 that senses skin stretching, a Meissner's corpuscle RA1 that senses weak and slow vibrations or friction as rapidly adapting, and a Pacinian corpuscle RA2 that senses strong and fast vibrations are illustrated. When a texture stimulation by a fine surface structure and a friction is applied, patterns of action potentials fired by each receptor may be different depending on a location and a type.

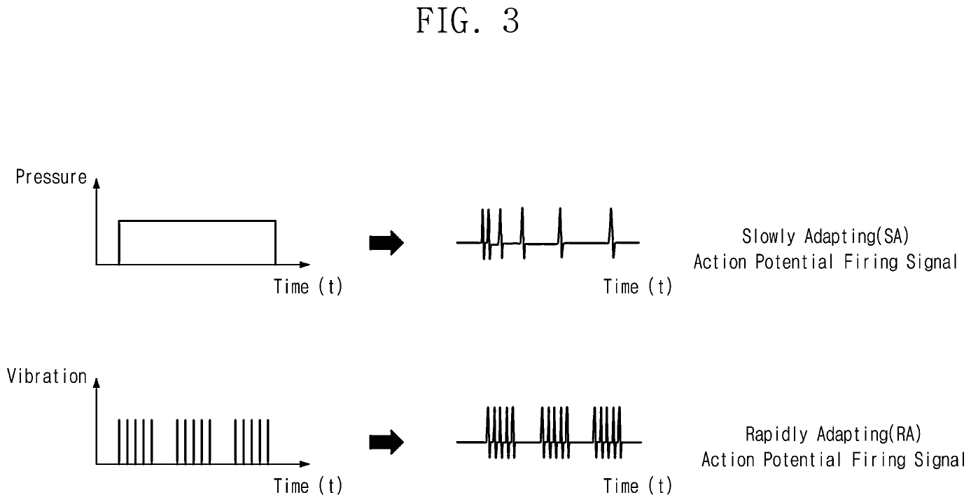

The Merkel cell SA1 or the Meissner's corpuscle RA1 distributed closest to the epidermis of the skin may generate a firing signal with their own unique signal characteristic in response to fine touch or pressure stimulation. The Ruffini corpuscle SA2 and the Pacinian corpuscle RA2 distributed inside the epidermis of the skin may generate a firing signal with their own unique signal characteristic in response to a skin stretching by strong touch or strong pressure or a strong and fast vibration stimulation. A more detailed description of this will be described later with reference to FIGS. 3 to 6.

In some embodiments, the stimulation threshold value of the Merkel cell SA1 is a value having a frequency of less than 100 Hz and an amplitude of less than 1 μm in a sine wave, and in particular, the Merkel cell SA1 is sensitive to frequencies of 50 Hz or more and 60 Hz or less. In contrast, the stimulation threshold value of the Pacinian corpuscle RA2 is a value having a frequency of less than 50 Hz and an amplitude of more than 100 μm in the sine wave, and in particular, the Pacinian corpuscle RA2 is sensitive to amplitudes of the order of 1 μm in frequencies of 250 Hz or more and 300 Hz or less. The stimulation threshold value of the Meissner's corpuscle RA1 is similar to that of the Merkel cell SA1 in that it is sensitive to frequencies of 50 Hz or more and 60 Hz or less in the sine wave, but a minimum stimulation threshold value has an amplitude of 5 μm or more. Like this, the frequency ranges of a sine wave stimulation that responds to the mechanoreceptors are different, and there are differences in the threshold value. In addition, since the mechanoreceptors have different spatial distributions in a body, it is possible to selectively stimulate the mechanoreceptors and to fire the action potential by controlling the waveform and the amplitude of the displacement stimulation when the micro-displacement is stimulated.

FIG. 3 is a diagram illustrating action potentials that are fired when pressure stimuli or vibration stimuli are applied to a skin in a slowly adapting and a rapidly adapting. Referring to FIG. 3, when pressure stimuli and vibration stimuli are applied to the skin, firing signals of the action potentials of the slowly adapting SA and the rapidly adapting RA are described.

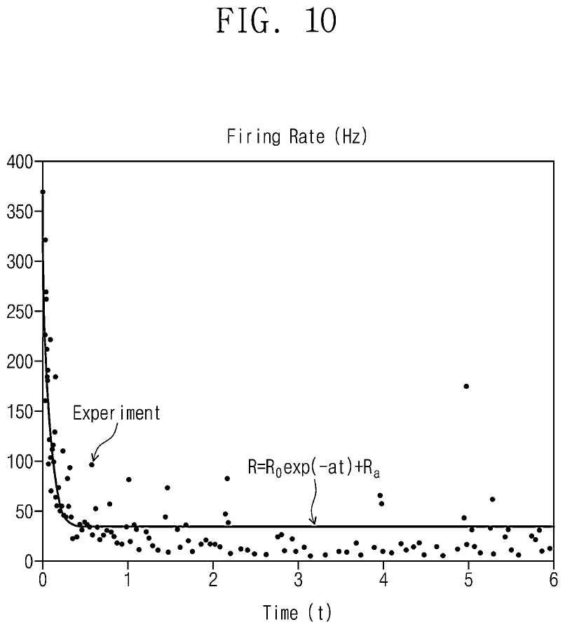

When a pressure stimulation of a uniform magnitude is applied to the skin for a specific period of time, the slowly adapting SA (e.g., the Merkel cell) exhibits a firing pattern with a fast firing rate at the moment when the pressure is applied, and as time passes, the firing rate decreases in an exponential form, so that a spike pattern with a slow firing rate may be maintained for a specific period of time. A more detailed description of this will be described later with reference to FIG. 10.

When a vibration stimulation of a uniform amplitude and a uniform frequency is applied to the skin, the rapidly adapting RA (e.g., the Meissner's corpuscle) exhibits a firing pattern with the same frequency as the vibration stimulation.

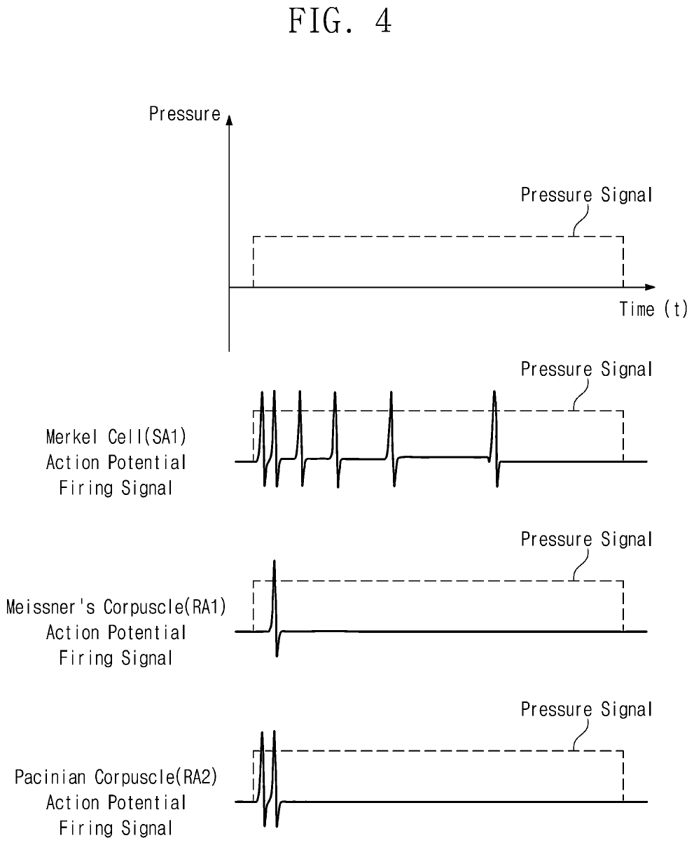

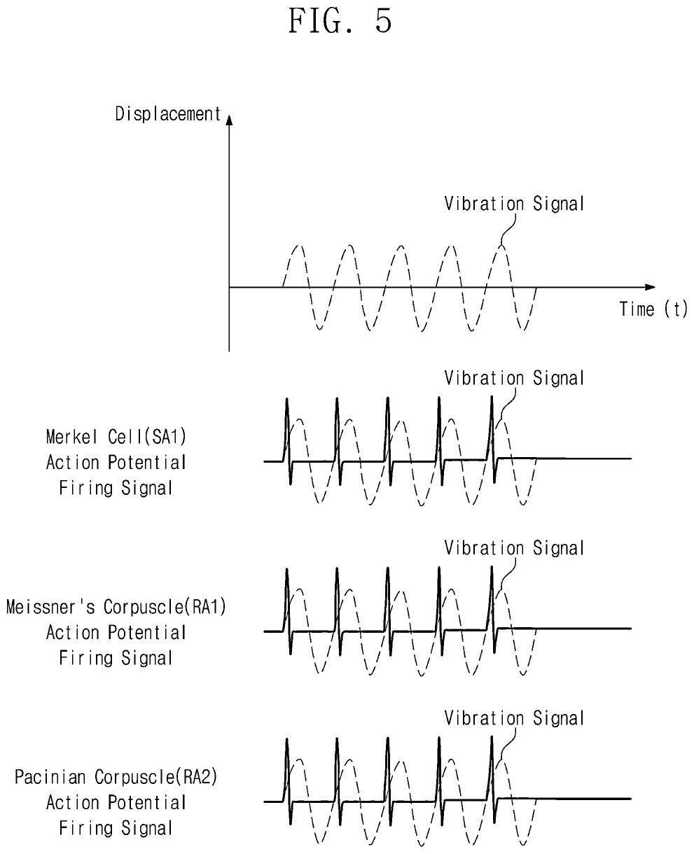

FIG. 4 is a diagram illustrating a firing of action potentials of a plurality of tactile receptors when pressure stimuli are applied to a skin, divided by Merkel cells, Meissner's corpuscles, and Pacinian corpuscles.

When a pressure stimulation of a specific magnitude is applied to the skin for a specific period of time, the following occurs. Initially, the action potential may be fired immediately in the Pacinian corpuscle RA2 and the Meissner's corpuscle RA1, which are the rapidly adapting RA. The Merkel cell SA1, which is the slowly adapting SA, has a firing signal that initially has a fast firing rate and then exponentially slows down as time passes. When the pressure stimulation is maintained for a specific period of time, the rapidly adapting RA (RA1 and RA2) no longer fire spikes, and only slowly adapting SA such as the Merkel cell SA1 may fire spikes. This slow firing has the characteristic of lasting for several minutes or more.

In proportion to the magnitude of the applied pressure, the Merkel cell SA1 has the characteristic of having a faster firing rate. In the case of the Ruffini corpuscle, which is the slowly adapting SA, the distribution density in the skin is low and the Ruffini corpuscle does not play a major role in sensing a sense of pressure, so it is not illustrated, but the scope of the present disclosure is not limited thereto.

FIG. 5 is a diagram illustrating a firing of action potentials of a plurality of tactile receptors when vibration stimuli are applied to a skin, divided by Merkel cells, Meissner's corpuscles, and Pacinian corpuscles.

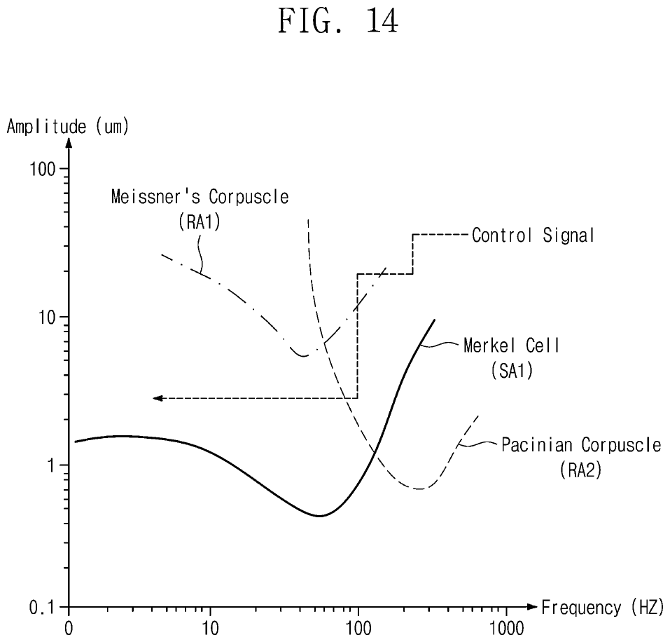

The Merkel cell SA1, the Meissner's corpuscle RA1, and the Pacinian corpuscle RA2 may fire action potentials with the same period as the period of the vibration stimulation when a vibration stimulation of a specific period is applied to the skin. The tactile receptors have the characteristic of firing selectively depending on the frequency and the amplitude of the vibration stimulation. A more detailed description of this will be described later with reference to FIG. 14.

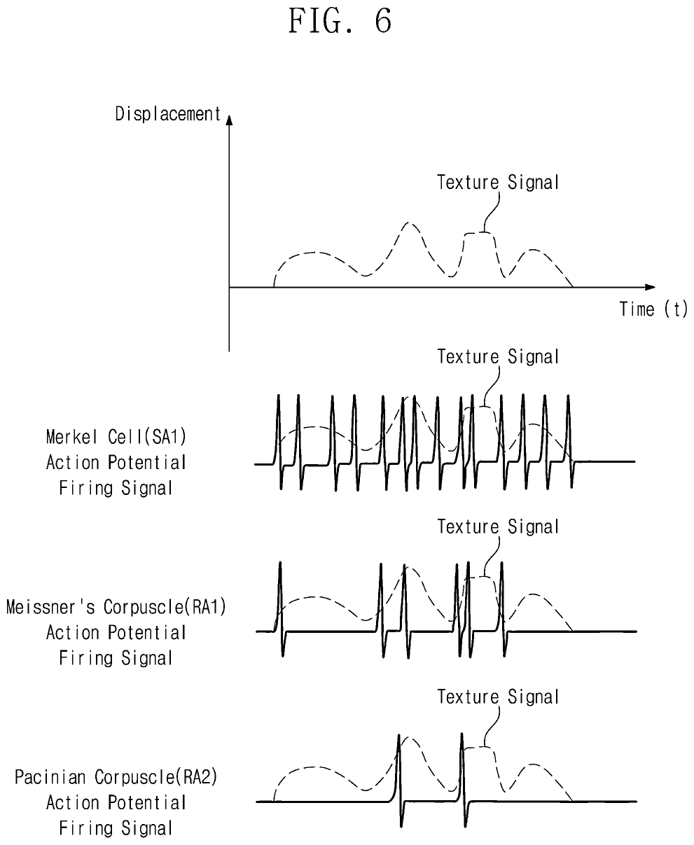

FIG. 6 is a diagram illustrating a firing of action potentials of a plurality of tactile receptors when a specific texture stimulation is applied to a skin, divided by Merkel cells, Meissner's corpuscles, and Pacinian corpuscles. Referring to FIG. 6, the firing patterns of the action potential spikes of the tactile receptors are described when a texture stimulation generated by rubbing the fine surface structure on the skin is applied.

When the skin contacts a soft surface, that is, when the texture signal over time is close to a curved shape, the action potential of the Meissner's corpuscle RA1 may fire a short spike. In the Merkel cell SA1, a pattern of continuous firing may appear when a pressure stimulation is applied to the skin.

When the skin contacts a rough surface, the action potential of the Pacinian corpuscle RA2 as well as the Meissner's corpuscle RA1 may fire briefly. In the Merkel cell SA1, a pattern of persistent spike firing may appear when a pressure stimulation is applied to the skin. The brain of FIG. 1 may distinguish the texture stimulation by combining the information of action potential spike signal patterns fired from each tactile receptor.

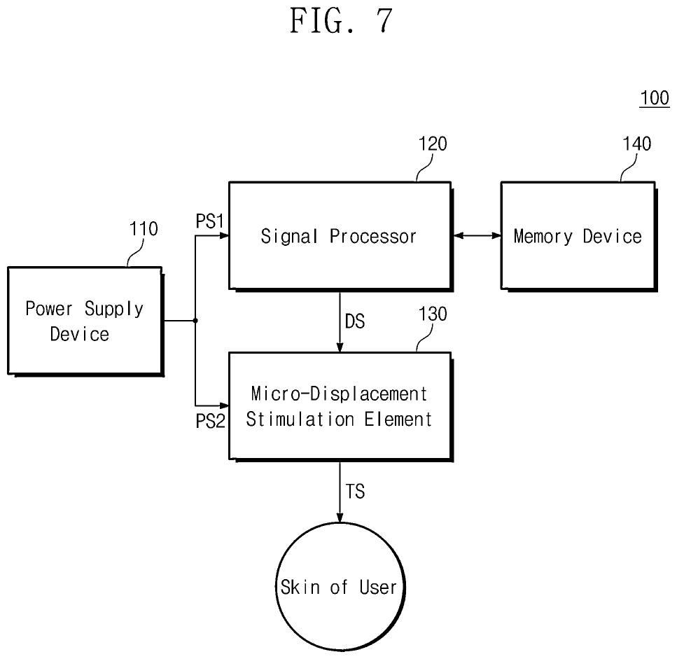

FIG. 7 is a diagram illustrating a virtual tactile implementation device 100, according to some embodiments of the present disclosure. Referring to FIG. 7, the virtual tactile implementation device 100 may include a power supply device 110, a signal processor 120, a micro-displacement stimulation element 130, and a memory device 140.

The power supply device 110 may be configured to supply electrical energy PS1 and PS2 to the signal processor 120 and the micro-displacement stimulation element 130. For example, the power supply device 110 may provide the first electrical energy PS1 to the signal processor 120. The power supply device 110 may provide the second electrical energy PS2 to the micro-displacement stimulation element 130.

The signal processor 120 may include at least one of hardware, software, or a combination of hardware and software. The signal processor 120 may provide a displacement signal DS to the micro-displacement stimulation element 130. The displacement signal DS may refer to a frequency and a waveform of the displacement stimulation (i.e., a tactile stimulation TS) applied to the skin by the micro-displacement stimulation element 130. The frequency and the waveform of the displacement signal DS may vary over time. Unlike a vibration signal having a uniform frequency and a uniform waveform, the displacement stimulation may refer to a signal having a variable frequency and a variable waveform over time.

The micro-displacement stimulation element 130 may provide a tactile stimulation to the skin based on the second electrical energy PS2 received from the power supply device 110 and the displacement signal DS received from the signal processor 120. The micro-displacement stimulation element 130 may selectively fire the action potentials of the plurality of tactile receptors according to the frequency and the waveform indicated by the displacement signal DS, and may provide a sense of pressure or a sense of texture by varying a time interval at which the action potentials of the plurality of tactile receptors are fired.

In some embodiments, the micro-displacement stimulation element 130 may be a piezoelectric actuator element. For example, the displacement of the micro-displacement element may be in the range of 100 μm or less. The micro-displacement element may apply a vibration signal having a waveform of a pulse signal or a sine wave signal with varying pulse width.

In some embodiments, the micro-displacement stimulation element 130 may apply a displacement stimulation that is a sine wave vibration with a pulse width of 1 msec or more and an amplitude of 100 μm or less to a user's skin.

In some embodiments, one or more micro-displacement stimulation elements 130 may be attached or adhered to a ring, a thimble, or a glove in an array structure. For example, the micro-displacement stimulation element 130 may be a stacked piezoelectric actuator or an element having a mechanism in which vertical displacement is amplified by attaching a spring, a bow or a cymbal to the stacked piezoelectric actuator.

The memory device 140 may include a plurality of tactile types and a plurality of displacement signal information. Each of the plurality of tactile types may include the magnitude of pressure and the degree of roughness. Each of the plurality of displacement signal information may include a waveform value and a frequency value of the displacement signal corresponding to one of the plurality of tactile types. The signal processor 120 may refer to the memory device 140 based on information about the tactile sense that the virtual tactile implementation device 100 intends to implement. The signal processor 120 may select one of the plurality of tactile types and may control the waveform and the frequency of the displacement signal over time depending on the displacement signal information corresponding to the tactile type.

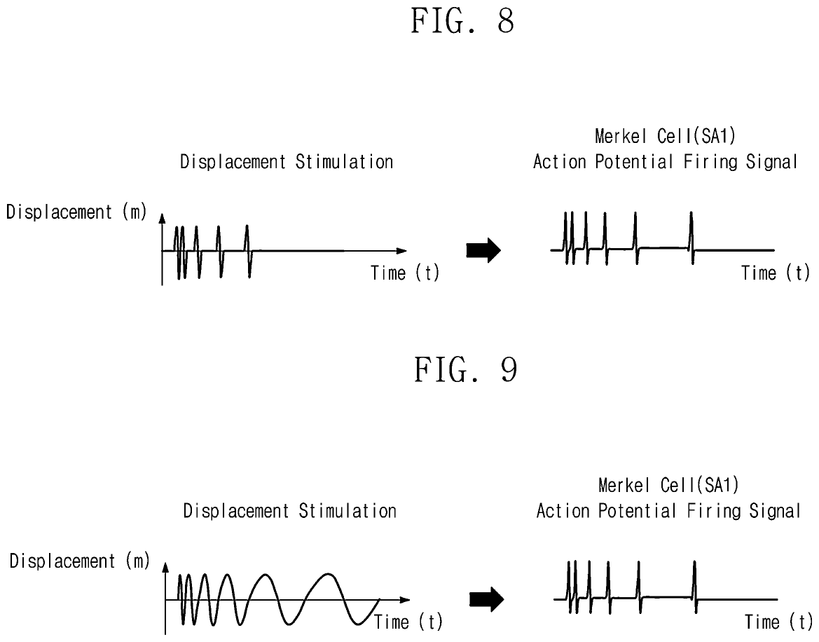

FIG. 8 is a diagram illustrating a firing signal of an action potential of the Merkel cell SA1 when a displacement stimulation is applied to a skin, according to some embodiments of the present disclosure. FIG. 8 illustrates a firing signal of the action potential of the Merkel cell SA1 when a micro-displacement stimulation element applies a pulse-shaped displacement stimulation to the skin depending on a displacement signal having a pulse-shaped waveform provided from the signal processor 120 of FIG. 7. Under the condition that firing of the action potential spike is induced in the Merkel cell SA1 only when a displacement stimulation is applied, a displacement stimulation having a pattern similar to that when a pressure stimulation is applied to the skin may be applied. Referring to FIG. 8, a spike firing pattern of the action potential of the Merkel cell SA1 similar to that when a pressure stimulation is applied to the skin in FIG. 4 is illustrated by the micro-displacement stimulation element 130 of FIG. 7. A horizontal axis of a displacement stimulation graph may indicate a time, and a vertical axis may indicate displacement. A horizontal axis of a firing signal graph may indicate a time, and a vertical axis may indicate potential.

The signal processor may control the displacement signal such that the waveform of the displacement signal includes at least one of a sine wave and a rectangular wave. The waveform of the displacement signal may be any shape, including a sine wave or a rectangular wave. A signal processor may control the displacement signal such that at least one of an amplitude size, a pulse width, and a duration of the displacement signal varies over time, thereby selectively inducing action potential spikes of the mechanoreceptors to fire. The signal processor may control a time interval between pulses of the displacement signal to generate a pattern of action potential firing times of the tactile receptors that appears when pressure or texture is applied to the skin.

FIG. 9 is a diagram illustrating a firing signal of an action potential of the Merkel cell SA1 when a displacement stimulation having a different pulse width from the displacement stimulation of FIG. 8 is applied to a skin. FIG. 9 is similar to FIG. 8, but the displacement stimulation having a sine waveform may be applied to the skin through the micro-displacement stimulation element according to the displacement signal having a sine waveform provided from the signal processor. Under the condition that the action potential spike is induced in the Merkel cell SA1 only when the micro-displacement stimulation is applied, the firing of the action potential spike of the Merkel cell SA1 may be induced by applying a displacement stimulation having a pattern similar to that when a pressure stimulation is applied to the skin. As described above in FIG. 8, by controlling the magnitude of the amplitude or the pulse width of the displacement signal, the signal processor may allow the micro-displacement stimulation element to selectively fire the action potentials of the mechanoreceptors, and by controlling the time intervals between the pulses of the action potential firing signal, the action potential firing time patterns of the tactile receptors that appear when pressure and texture are applied to the skin may be generated.

FIG. 10 is a diagram illustrating a firing rate curve representing a firing rate of an action potential of the Merkel cell over time when a pressure stimulation is applied to the skin. A horizontal axis may indicate a time, and a vertical axis may indicate a firing rate. Referring to FIG. 10, according to some embodiments, when a pressure stimulation is applied to the skin of a human or an animal, a spike firing rate of hundreds of hertz (Hz) or more is observed in the initial Merkel cell in proportion to the magnitude of the pressure, and accordingly, the time interval between spikes (ISI; Inter Spike Interval) is on the order of several milliseconds (msec). However, over time, within approximately 0.3 seconds, the spike firing rate decays exponentially, and after approximately 0.3 seconds, the spike firing rate drops to tens of hertz, and accordingly, the time interval between spikes may increase to tens of milliseconds. After that, a pattern appears in which the firing rate and the time interval between spikes are maintained.

For example, a firing rate curve “R” indicates the neural spike firing rate in the Merkel cell SA1 over time when a pressure stimulation of 100 mN is applied to a cylindrical probe with a diameter of 1 mm for a specific period of time. The firing rate curve “R” may refer to an example of fitting the time-dependent decay curve of the firing rate using an exponential decay function without considering the phenomenon of irregular changes in the firing rate. A graph represented by dots may be an example of experimental results.

In detail, the firing rate curve “R” may have a shape in which the firing rate rapidly decreases exponentially over time and then remains uniform after a specific period of time. In this case, the firing rate curve “R” may be expressed by Equation R₀*exp(−at)+Rₐ. In this time, R₀ may indicate an amplitude of an exponential function, “a” may indicate an attenuation coefficient, and Rₐ may indicate a convergent firing rate that remains uniform after a specific period of time. In some embodiments, the firing rate curve “R” may be derived from a minimum deviation method.

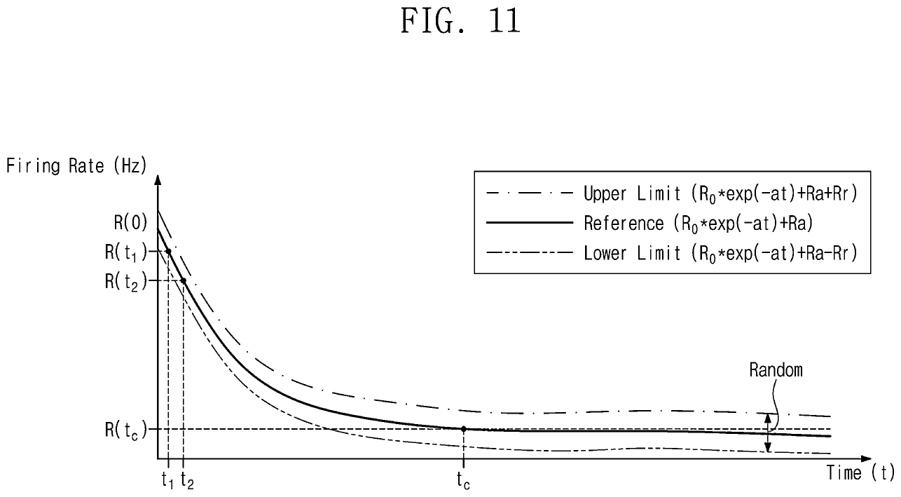

FIG. 11 is a diagram illustrating artificially generating the firing rate curve “R” of FIG. 10 to be similar to an experimental value, according to some embodiments of the present disclosure. Referring to FIG. 11, a reference firing rate curve, an upper limit firing rate curve, and a lower limit firing rate curve are respectively illustrated. A horizontal axis may indicate a time, and a vertical axis may indicate a firing rate. The reference firing rate curve may be the same as the firing rate curve “R” of FIG. 10, and may be expressed by Equation R₀*exp(−at)+Rₐ. The lower limit firing rate curve may be the same as the reference firing rate curve shifted parallel to the vertical axis direction, and may be expressed by Equation R₀*exp(−at)+Rₐ-Rᵣ. In this case, Rᵣ may refer to a random constant, and the method of obtaining Rᵣ will be described below. The upper limit firing rate curve may be the same as the reference firing rate curve shifted parallel to the vertical axis direction, and may be expressed by Equation R₀*exp(−at)+Rₐ+Rᵣ. In this case, Rᵣ may refer to a random constant, and the method of obtaining Rᵣ will be described below.

In detail, in a first time interval (e.g., t≤t_{c}), the firing rate curve may have an exponential decay over time. After the first time interval, in a second time interval (e.g., t>t_{c}), the firing rate curve may increase and decrease irregularly over time. For the later interval where the firing rate increases and decreases irregularly, the firing rate curve may be adjusted to be similar to the action potential spike firing rate of the experimental tactile receptors by using a random function having a specific range (−Rᵣ or more and Rᵣ or less).

In some embodiments, during the second time interval, the firing rate curve may have a random deviation within a range of the second value Rᵣ less than the first value based on the first value (e.g., R(t_{c})).

In some embodiments, the signal processor of FIG. 7 may control the time interval at which the action potential fires such that the firing rate decreases or increases over time. In addition, the signal processor may provide the user with a sense of pressure that the pressure changes over time by the micro-displacement stimulation element by controlling the decrease speed or the increase speed.

For example, a first time difference (t₁=1/R(0)) may be obtained by using the initial firing rate R(0) when “t” is “0” from the reference firing rate curve. In addition, the first time difference t₁ may be substituted into the reference firing rate curve to obtain the first firing rate R(t₁). Also, a second time difference (t₂=t₁+1/R(t₁)) may be obtained using the first firing rate R(t₁). Furthermore, an n-th time difference and an n-th firing rate may be obtained. In this case, “n” may be any natural number. In detail, the time intervals between spikes may be obtained sequentially using an iteration method.

As a detail example, the reference time t_{c} may refer to the time when the firing rate drops to 1/100 of the initial firing rate R(0) when “t” is “0”. In addition, when the attenuation coefficient “a” of the exponential attenuation is 17.28, the reference time t_{c} may be 0.1157 seconds.

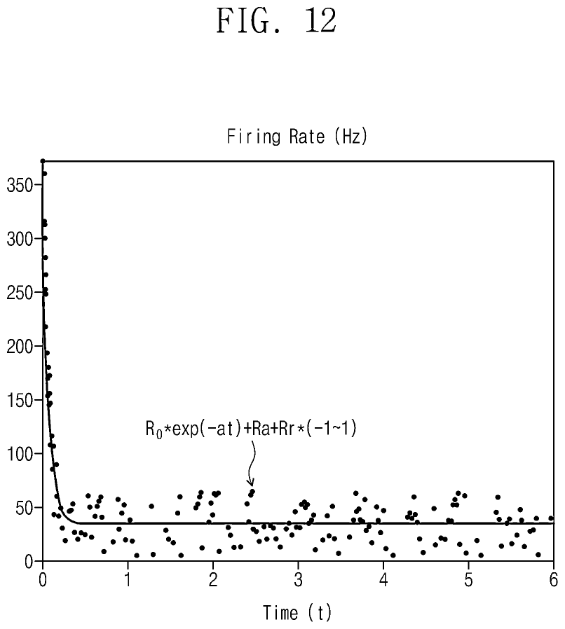

FIG. 12 is a diagram illustrating a firing rate curve graph generated by a method of FIG. 11. Referring to FIG. 12, an example of a firing signal of an action potential of the tactile receptors having a random firing rate pattern within a specific range (e.g., −Rᵣ<R<+Rᵣ) centered around a value (e.g., convergent firing rate Rₐ) that initially decays exponentially and then converges later is illustrated. A horizontal axis may indicate a time, and a vertical axis may indicate a firing rate.

As illustrated in FIG. 10, which is an example of the firing rate of an action potential spike obtained by applying a pressure stimulation to a tactile receptor, a pattern very similar to a signal pattern may be obtained in which an initial exponential decay is rapid and then, after a specific period of time, the firing rate changes randomly within a specific range centered on a uniform firing rate.

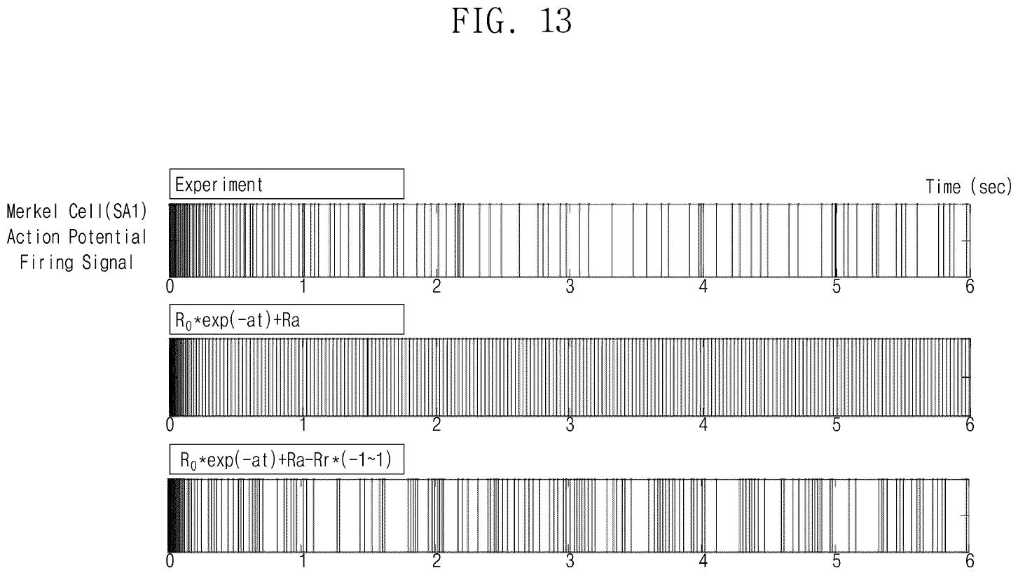

FIG. 13 is a diagram illustrating a firing signal of an action potential of the Merkel cell SA1 over time, according to some embodiments of the present disclosure. Referring to FIG. 13, an experimental graph (Experiment) illustrating the timing at which the action potential spikes of the Merkel cell SA1 obtained by applying a pressure stimulation illustrated in FIG. 10 to the skin are fired over time, a firing time graph of the firing rate curve (R₀*exp(−at)+Rₐ) that has the characteristic of initially exponential decay and then later converging to a uniform firing rate to simulate the experimental graph (Experiment), and a firing time graph of a firing rate curve (R₀*exp(−at)+Rₐ+Rᵣ(−1 to 1)) that has the characteristic of initially exponential decay and then later converging to a uniform value, but with a random firing rate component added within a specific range over the entire time, to more accurately simulate the action potential spike firing are respectively illustrated. When comparing the graphs, a signal pattern more similar to the experimental graph (Experiment) appears when a random firing rate component is added. Depending on an individual human or a living organism or on the applied pressure, R₀, a, Rₐ, and Rᵣ may vary. In an actual product, data obtained from an experiment may be referred to, but each value may be adjusted to adjust the intensity at which an actual person feels pressure, etc., and may be applied.

FIG. 14 is a graph illustrating conditions under which action potentials of the Merkel cell SA1, the Meissner's corpuscle RA1, and the Pacinian corpuscle RA2 are fired depending on a frequency and an amplitude of a displacement signal when a displacement signal having a sine wave waveform is used in some embodiments of the present disclosure. A horizontal axis may indicate a frequency, and a vertical axis may indicate an amplitude.

The Merkel cell SA1 with slow response characteristics fires a spike at the lowest amplitude of about 300 nm for a sine wave vibration of 30 Hz or more and 50 Hz or less, and has the property of firing the spike in a wide range of 1 Hz or more and 300 Hz or less when an amplitude of several micrometers (um) is applied. The Meissner's corpuscle RA1 with fast response characteristics fires a spike when an amplitude of 5 μm or more with respect to a sine wave vibration of 30 Hz or more and 50 Hz or less is applied, and has the characteristic that a spike is not fired with respect to a sine wave vibration with a frequency of 1 Hz or more and 500 Hz or less and an amplitude of 5 μm or less. The Pacinian corpuscle RA2, which have a faster response characteristic than the Meissner's corpuscle RA1, fires a spike at an amplitude of 1 μm or more when a sine wave vibration of 200 Hz or more and 300 Hz or less is applied, and has the characteristic that the spike is not fired when an amplitude of 1 μm or more and 2 μm or less at a frequency of 100 Hz or less is applied.

In addition, since the action potential has the characteristic of firing at a time interval equal to a period (i.e., single pulse width) of the sine wave to which the vibration of the displacement stimulation is applied, the time intervals between action potential firing spikes may be controlled by controlling the single pulse width of the vibration of the applied displacement stimulation.

Considering these characteristics, by changing the frequency of the sine wave vibration, as indicated by the dotted arrows in the graph, spikes may be selectively fired only in specific tactile receptors. For example, when applying the sine wave vibration with a frequency of 100 Hz or less and an amplitude of about 3 μm, spikes are not fired in rapid-response tactile receptors such as the Pacinian corpuscle RA2 or the Meissner's corpuscle RA1, and spikes may be selectively fired only in the Merkel cell SA1 that senses slow responses.

As another example, when applying the sine wave vibration with a frequency of 100 Hz or more and an amplitude of 10 μm or more and 20 μm or less, spikes may be fired in all tactile receptors SA1, RA1, and RA2. In addition, by further increasing the frequency from 300 Hz to 400 Hz or more and by further increasing amplitude size, spike firing in the Pacinian corpuscle RA2 may be better induced.

In addition, the time intervals between the action potential spikes of the tactile receptors may be controlled depending on the single pulse width of the applied sine wave displacement signal. Therefore, the action potentials of the tactile receptors may be selectively fired by controlling the single pulse width and the amplitude of the displacement stimulation. Furthermore, the time intervals at which the action potentials of the tactile receptors are fired may be controlled depending on the single pulse width of the displacement stimulation.

Using this method, the action potentials of the tactile receptors may be fired by the displacement stimulation in a pattern similar to the action potential firing signal pattern fired in each tactile receptor when a pressure stimulation is applied to the skin.

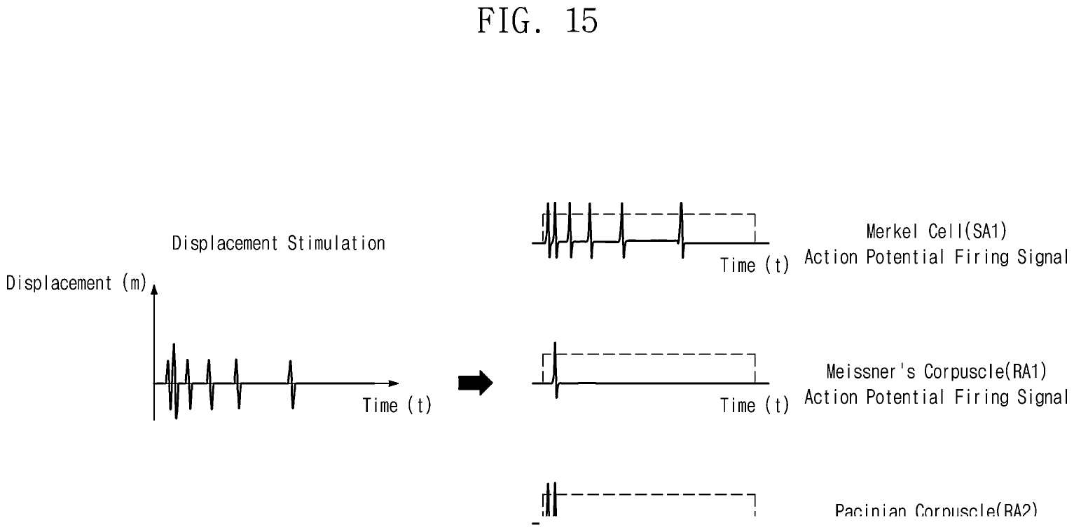

FIG. 15 is a diagram illustrating a firing of action potentials of a plurality of tactile receptors when a signal processor applies a displacement signal of a pulse signal and changes an amplitude of a displacement signal and a time interval between pulses of the displacement signal, according to some embodiments of the present disclosure. Referring to FIG. 15, when applying a pressure stimulation by a displacement stimulation according to a displacement signal of a pulse signal, each tactile receptor may fire in a similar way to the action potential spike pattern that fires in each tactile receptor.

Although the displacement signal is illustrated as a pulse signal, the displacement signal may be used in various patterns such as a sine wave or a rectangular wave pulse shape, and the present disclosure does not limit thereto.

In response to initially very short successive pulse included in the displacement stimulation, the action potential spike of the Pacinian corpuscle RA2 may fire. In a pulse with a large amplitude included in the displacement stimulation, the action potential spike may be fired up to the Meissner's corpuscle RA1. In response to a period with a wide time interval between pulses included in the displacement stimulation, the action potential spike may be fired only in the Merkel cell SA1.

When the signal processor controls the displacement signal such that the micro-displacement stimulation element applies a displacement stimulation such as that in FIG. 15 to the skin, the signal pattern in which action potential spikes are fired in the tactile receptors SA1, RA1, and RA2 may be very similar to the signal pattern in which the action potential spikes are fired in each of the tactile receptors when a pressure stimulation is applied in FIG. 4.

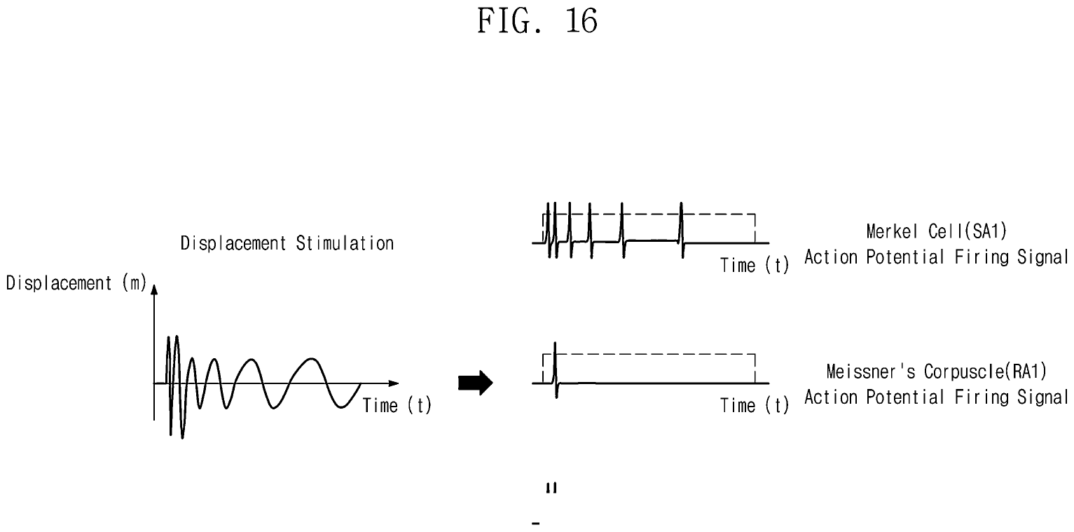

FIG. 16 is a diagram illustrating a firing of action potentials fired in a plurality of tactile receptors when a signal processor applies a displacement signal having a sine wave waveform and changes an amplitude and a pulse width of a displacement signal, according to some embodiments of the present disclosure.

The Pacinian corpuscle RA2 may be selectively fired in response to a sine wave displacement stimuli of a first frequency (e.g., 200 Hz or 5 msec) and a first amplitude (relatively large) included in the displacement stimulation. The Meissner's corpuscle RA1 may also be fired in response to sine wave displacement stimuli of a second frequency (e.g., 100 Hz or 10 msec) lower than the first frequency included in the displacement stimulation and a first amplitude (relatively large). In addition, only the Merkel cell SA1 may fire spikes in response to the sine wave displacement stimuli of a third frequency (e.g., tens to several hertz) relatively low and slow included in the displacement stimulation.

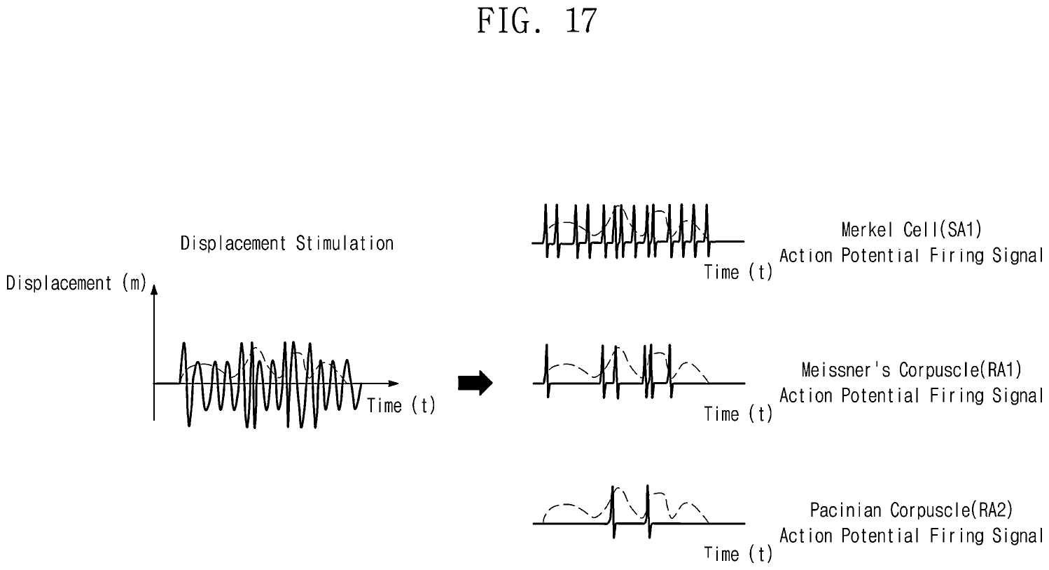

In detail, the virtual tactile implementation device may selectively fire the action potentials for each tactile receptor, and by controlling the time intervals at which the action potentials of the tactile receptors are fired, the pattern in which the action potential spikes are fired in each tactile receptor may be similar to the pressure stimulation of FIG. 4. FIG. 17 is a diagram illustrating a firing of action potentials of a plurality of tactile receptors when a signal processor applies a displacement signal having a sine wave waveform and changes an amplitude and a pulse width of a displacement signal, according to some embodiments of the present disclosure.

In response to a sine wave having a pulse width of a fast frequency included in the displacement stimulation and a displacement amplitude greater than 10 μm, spikes may be fired in both the Pacinian corpuscle RA2 and the Meissner's corpuscle RA1. In response to a sine wave having a frequency of 100 Hz (i.e., a pulse width of 10 msec) included in the displacement stimulation, spikes may be fired only in the Meissner's corpuscle RA1 and spikes may not be fired in the Pacinian corpuscle RA2. In addition, in response to a sine wave having a frequency of 100 Hz (i.e., a pulse width of 10 msec) included in the displacement stimulation and an amplitude lower than 10 μm, spikes may not be fired in the Meissner's corpuscle RA1 and spikes may be fired only in the Merkel cell SA1.

In some embodiments, the signal processor of FIG. 7 may allow the micro-displacement stimulation element to provide a virtual texture to the user's skin by controlling the displacement signal such that the pulse width of the displacement signal of the sine wave has two or more different pulse widths of 1 msec or more, or the amplitude of the displacement signal has two or more different sizes of 100 μm or less.

In this way, when the signal processor controls the pulse width and amplitude of the displacement signal, spikes may selectively be fired in the tactile receptors. The action potential firing signal pattern of the tactile receptors of FIG. 17 may be similar to the action potential firing signal pattern of the tactile receptors when a sense of texture is applied to the skin in FIG. 6. In detail, the virtual tactile implementation device may provide a virtual texture to the user by inducing a firing pattern similar to the action potential spike firing pattern of the tactile receptor that appears when a texture stimulation is applied.

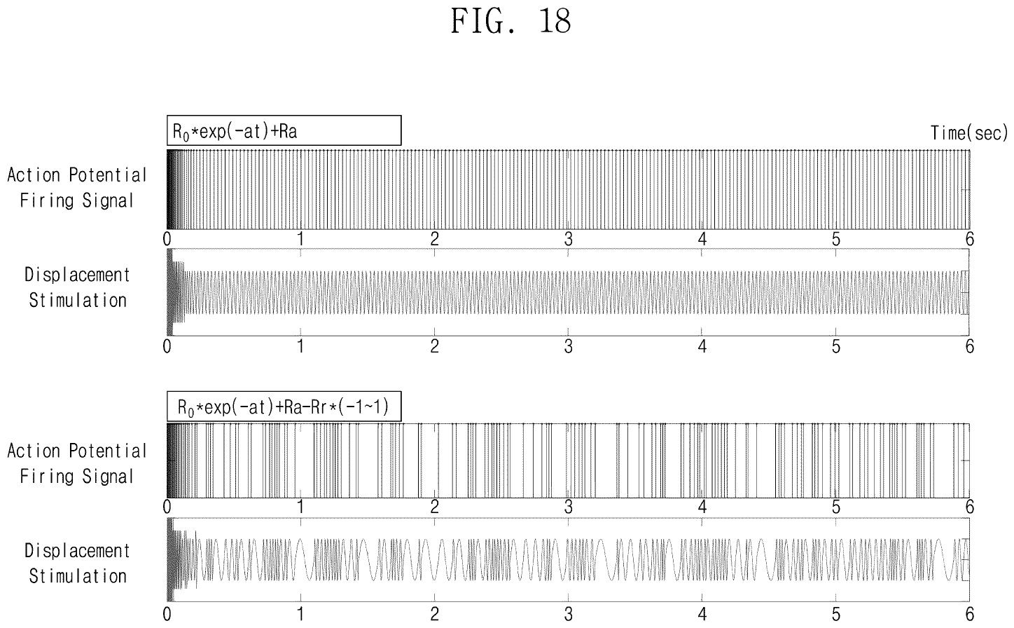

FIG. 18 illustrates an example of a displacement stimulation of a micro-displacement stimulation element generated by varying a pulse width of the displacement stimulation such that a firing similar to a time-dependent action potential spike firing pattern of a tactile receptor of the Merkel cell SA1 illustrated in FIG. 13 may be induced, according to some embodiments of the present disclosure. Referring to FIG. 18, firing rate graphs of the action potential firing signals of FIG. 13 (including Rₐ and Rᵣ components) and displacement stimuli corresponding to each of the action potential firing signals are illustrated. A horizontal axis may indicate a time.

In the firing rate graphs, in the very early stage when the firing rate is high, the signal processor may increase the amplitude of the displacement signal so that spike firing occurs quickly in the Pacinian corpuscle or the Meissner's corpuscle by the micro-displacement stimulation element, in the stage when the firing rate decreases, the signal processor may decrease the amplitude of the displacement signal so that spikes are mainly fired in the Merkel cells by the micro-displacement stimulation element, but spikes are occasionally fired in the Meissner's corpuscles, and in the later stage when the firing rate becomes very low, the signal processor may set the amplitude of the displacement signal to the lowest possible level so that spikes are fired only in the Merkel cells by the micro-displacement stimulation element.

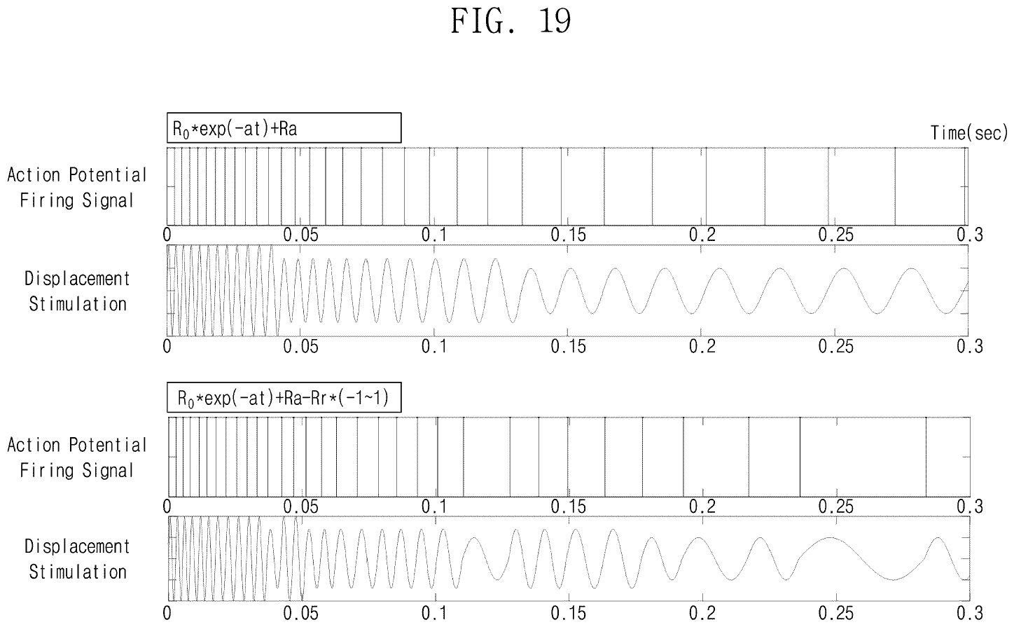

FIG. 19 is a diagram illustrating an enlarged view of an initial 0.3 second period of FIG. 18.

The signal processor may generate an action potential spike firing pattern in each of the tactile receptors similar to when a pressure stimulation is applied to the skin by the micro-displacement stimulation by controlling a displacement signal amplitude in conjunction with the time interval during which the displacement stimulation is applied to the skin or the size the firing rate. Therefore, the virtual tactile implementation device may provide a pressure stimulation to the skin, such as a finger, as if the pressure stimulation is applied to the skin.

The various types of pressure sensitivity that a person feels when touching an object may be different in the case of fast contact and slow contact, and the pressure sensitivity with changing pressure may be implemented using the device and method disclosed in the present disclosure, and a sense of texture when touching various objects may also be implemented.

For example, the signal processor may provide a sense of pressure that may distinguish two or more levels of pressure by the micro-displacement stimulation element by controlling the time interval during which the action potential of the Merkel cell is fired such that the firing rate of the action potential of the Merkel cell is attenuated over time, and by controlling the magnitude of the firing rate.

In detail, the signal processor may control the displacement signal such that a firing pattern similar to the action potential firing pattern of the Merkel cell appears when a first pressure magnitude is applied to the skin, and then may control the displacement signal such that a firing pattern similar to the action potential firing pattern of the Merkel cell appears when a second pressure magnitude different from the first pressure magnitude is applied to the skin.

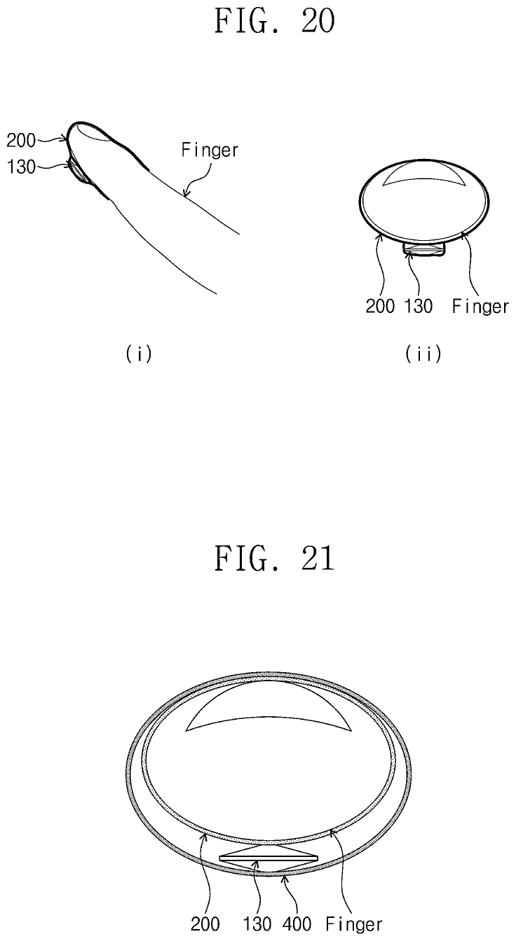

FIG. 20 is a side view and a front view of a contact device 200 and the micro-displacement stimulation element 130, which are attached to a finger, according to some embodiments of the present disclosure. Referring to a side view “i” and a front view “ii”, the contact device 200 is in close contact with the skin, and allows the micro-displacement stimulation element to be closely contacted with the skin so that the displacement stimulation is in close contact with the skin.

In some embodiments, the contact device 200 may be in the form of a flexible thimble or a flexible glove.

In some embodiments, the micro-displacement stimulation element may be a piezoelectric actuator element. The piezoelectric actuator element may provide a rapid displacement stimulation of several hundred hertz or more, and since the displacement is very small, within 1 um, a bow or a cymbal structure may be used or an amplifying structure such as a spring may be used together to increase the displacement to the micrometer level.

For example, a bow or a cymbal structure may be bonded to both sides of the piezoelectric actuator element to provide a micrometer-sized displacement.

FIG. 21 is a front view of a ring-shaped contact device 400 including the micro-displacement stimulation element 130 inside, according to some embodiments of the present disclosure.

The ring-shaped contact device 400 may refer to a rigid ring additionally worn outside the contact device 200 worn on the finger of FIG. 20. The ring-shaped contact device 400 may apply micro-displacement stimulation by contacting the micro-displacement stimulation element 130 attached inside to the skin of the finger. According to some embodiments, the micro-displacement stimulation element 130 attached inside the rigid ring without the contact device 400 may directly stimulate the skin of the finger.

In some embodiments, the micro-displacement stimulation element may be arranged in a one-dimensional array form or a two-dimensional array form. The micro-displacement stimulation element may provide a realistic pressure sense to the skin by applying a displacement stimulation with the controlled frequency and the controlled amplitude with respect to the tactile receptors distributed in the skin of the finger to generate different spike signals for each tactile receptor. In addition, a waveform, a frequency, and an amplitude of the displacement stimulation may be mixed over time to provide a sense of pressure, a sense of vibration, and even a sense of texture.

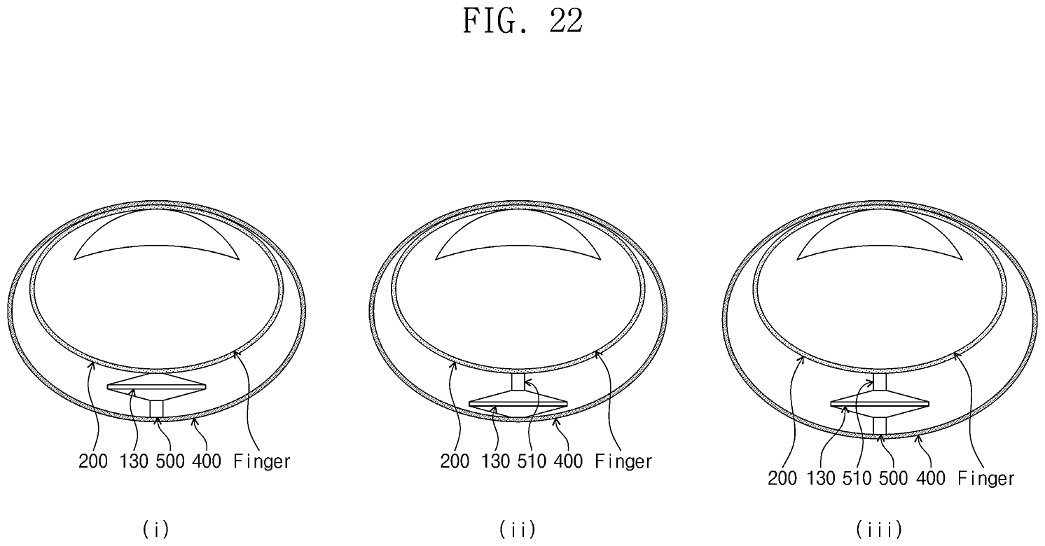

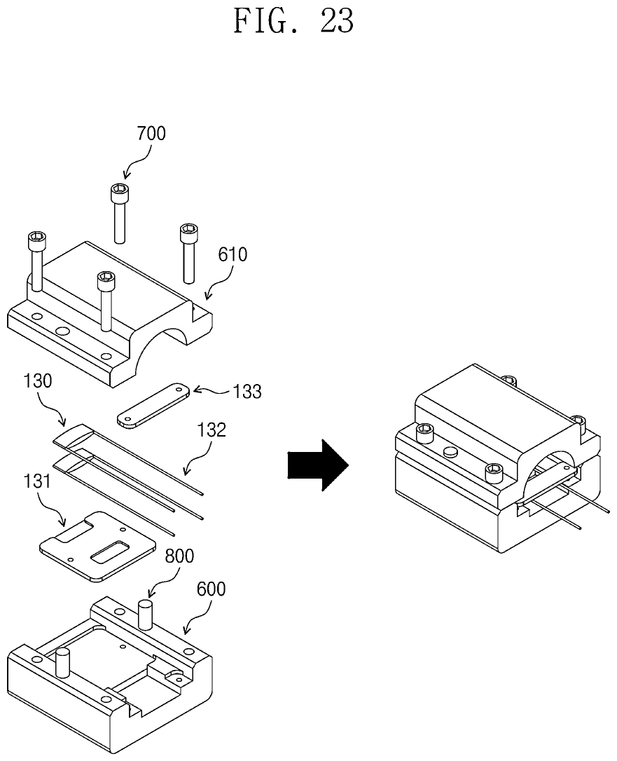

FIG. 22 is a diagram illustrating a contact device including a protrusion in a front view of FIG. 21.

The contact device 400 may further include protrusions 500 and 510 that allow the micro-displacement stimulation element 130 worn on the finger to be in better contact with the skin of the finger. The protrusions 500 and 510 may be used by being bonded between the ring-shaped contact device 400 and the cymbal structure of the micro-displacement stimulation element 130 or by being bonded to the cymbal of the micro-displacement stimulation element that comes into contact with the skin of the finger.

The ring-shaped contact device 400 may generally be made of various metals. For example, the ring-shaped contact device 400 may be made of various materials such as ceramic or plastic, as long as it may apply pressure to the skin of the finger, the scope of the present disclosure is not limited thereto, and may be used with various structures such as a band or a tie as well as the ring structure.

FIG. 23 is a diagram illustrating a contact device, according to some embodiments of the present disclosure. Referring to FIG. 23, a design diagram of the micro-displacement stimulation element 130 and a contact device for applying pressure to the skin is illustrated such that the micro-displacement stimulation element 130 may be more strongly contacted to the skin of the finger, as in FIG. 22.

In some embodiments, the micro-displacement stimulation element 130 is arranged on a micro-displacement stimulation element substrate 131 in a 1×2 array form, and micro-displacement stimulation may be applied through micro-displacement stimulation element wirings 132. A wiring fixing unit 133 of the micro-displacement stimulation element 130 may fix the micro-displacement stimulation element wirings 132. To control the pressure with which the micro-displacement stimulation element 130 is adhered to the skin of the finger, a lower holder 600, an upper holder 610, a pressure adjustment screw 700, and a holder tilt fixing shaft 800 may be used. The pressure adjustment screw 700 and the holder tilt fixing shaft 800 are used to adjust a gap between the lower holder 600 and the upper holder 610.

The virtual tactile implementation device according to the present disclosure may have a built-in battery corresponding to the power supply device 110 of FIG. 7, and may provide a user with a sense of pressure or a sense of texture when a screen or a space is touched by linking with a smartphone, a touchscreen, or VR (virtual reality) glasses. Alternatively, when clicking on an object or an application on the screen or the space, the virtual tactile implementation device may provide a user with a sense of pressure, such as when pressing a button, or may provide a user with a virtual tactile sensation, such as the texture or the friction of an object, when the user touches or rubs against the object. Furthermore, the virtual tactile implementation device may be additionally provided with a communication unit (e.g., a BLE (Bluetooth Low Energy) device, a Wi-Fi device, etc.) that may exchange information such as the location and presence of touch with respect to a display or VR glasses and the tactile stimulation device. In addition, a virtual sense of touch may be implemented by arranging the micro-displacement stimulation element 130 on the display surface pad in an array form, and the present disclosure is not limited thereto.

According to an embodiment of the present disclosure, a virtual tactile implementation device that selectively stimulates mechanoreceptors and a method of operating the same are provided.

In addition, a device and a method for implementing virtual tactile senses such as high-resolution pressure senses and various textures are provided by selectively stimulating each of various mechanoreceptors existing in the skin layer through adjusting of a micro-displacement signal to fire action potentials and by controlling a time interval at which the action potential is fired.

The above descriptions are detail embodiments for carrying out the present disclosure. Embodiments in which a design is changed simply or which are easily changed may be included in the present disclosure as well as an embodiment described above. In addition, technologies that are easily changed and implemented by using the above embodiments may be included in the present disclosure. Therefore, the scope of the present disclosure should not be limited to the above-described embodiments and should be defined by not only the claims to be described later, but also those equivalent to the claims of the present disclosure.

What is claimed is:

1. A virtual tactile implementation device comprising:
one or more micro-displacement stimulation elements in close contact with a skin;
a signal processor configured to apply a displacement signal to the micro-displacement stimulation element; and
a power supply device configured to supply electrical energy to the micro-displacement stimulation element and the signal processor, and
wherein the signal processor is configured to control a waveform and an amplitude of the displacement signal to allow the micro-displacement stimulation element to selectively fire action potentials of a plurality of tactile receptors within the skin, and to control a time interval at which each of the action potentials is fired to allow the micro-displacement stimulation element to provide a sense of pressure or a sense of texture.

2. The virtual tactile implementation device of claim 1, wherein the signal processor is configured to control the waveform and the amplitude of the displacement signal to allow the micro-displacement stimulation element to fire only the action potential of a Merkel cell among the plurality of tactile receptors, and to control a time interval at which the action potential of the Merkel cell is fired to allow the micro-displacement stimulation element to provide the sense of pressure or the sense of texture.

3. The virtual tactile implementation device of claim 2, wherein the signal processor is configured to control the time interval at which the action potential of the Merkel cell is fired such that a firing rate of the action potential of the Merkel cell attenuates over time, and to control a magnitude of the firing rate to allow the micro-displacement stimulation element to provide the sense of pressure capable of distinguishing between two or more levels of pressure.

4. The virtual tactile implementation device of claim 2, wherein the signal processor is configured to control the time interval such that a firing rate of the action potential exponentially decays over time during a first time period, and to control the time interval such that the firing rate randomly deviates within a range of a second value less than a first value centered on the first value during a second time period after the first time period, to allow the micro-displacement stimulation element to provide the sense of pressure.

5. The virtual tactile implementation device of claim 2, wherein the signal processor is configured to control the time interval such that the firing rate is attenuated or increased over time, and to control an attenuation speed or an increase speed to allow the micro-displacement stimulation element to provide the sense of pressure in which a pressure changes over time.

6. The virtual tactile implementation device of claim 1, wherein the signal processor is configured to control the displacement signal to allow the micro-displacement stimulation element to provide the sense of texture such that a pulse width of the displacement signal has two or more different pulse widths of 1 msec or more, or an amplitude of the displacement signal has two or more different magnitudes of 100 um or less, and the waveform of the displacement signal becomes a sine wave.

7. The virtual tactile implementation device of claim 1, wherein the signal processor is configured to control the displacement signal to allow the micro-displacement stimulation element to selectively fire spikes of the action potential of the plurality of tactile receptors such that the displacement signal is a pulse signal and at least one condition of the amplitude of the displacement signal and a time interval between pulses of the displacement signal changes over time.

8. The virtual tactile implementation device of claim 1, wherein the waveform of the displacement signal includes a sine wave and a rectangular wave.

9. The virtual tactile implementation device of claim 1, wherein the signal processor is configured to control the displacement signal to allow the micro-displacement stimulation element to selectively fire spikes of the action potential of the plurality of tactile receptors such that the displacement signal is a sine wave signal and at least one condition of a magnitude of the amplitude and a pulse width of the displacement signal changes over time.

10. The virtual tactile implementation device of claim 1, further comprising:
a contact device configured to contact the skin with the micro-displacement stimulation element, and
wherein the contact device is at least one of an elastic thimble structure, a ring structure, and a glove structure.

11. The virtual tactile implementation device of claim 1, further comprising:
a contact device configured to contact the skin with the micro-displacement stimulation element, and
wherein the contact device includes at least one of an elastic band and a screw which adjust a contact pressure between the micro-displacement stimulation element and the skin.

12. The virtual tactile implementation device of claim 1, wherein the micro-displacement stimulation element is an element capable of applying a sine wave vibration having a pulse width of 1 msec or more and an amplitude of 100 um or less to the skin.

13. The virtual tactile implementation device of claim 1, wherein the micro-displacement stimulation element is a piezoelectric actuator element.

14. A method of operating a virtual tactile implementation device including one or more micro-displacement stimulation elements which are in close contact with a skin and a signal processor, the method comprising:
controlling, by the signal processor, a waveform and an amplitude of a displacement signal so as to be provided to the micro-displacement stimulation element; and
providing, by the micro-displacement stimulation element, a tactile stimulation to the skin depending on the displacement signal, and
wherein the signal processor controls the waveform and the amplitude of the displacement signal to allow the micro-displacement stimulation element to selectively fire action potentials of a plurality of tactile receptors within the skin, and controls a time interval at which each of the action potentials is fired to allow the micro-displacement stimulation element to provide a sense of pressure or a sense of texture.

15. The method of claim 14, wherein the signal processor controls the waveform and the amplitude of the displacement signal to allow the micro-displacement stimulation element to fire only the action potential of a Merkel cell among the plurality of tactile receptors, and controls a time interval at which the action potential of the Merkel cell is fired to allow the micro-displacement stimulation element to provide the sense of pressure or the sense of texture.

16. The method of claim 15, wherein the signal processor controls the time interval at which the action potential of the Merkel cell is fired such that a firing rate of the action potential of the Merkel cell attenuates over time, and controls a magnitude of the firing rate to allow the micro-displacement stimulation element to provide a sense of virtual pressure capable of distinguishing between two or more levels of pressure.

17. The method of claim 15, wherein the signal processor controls the time interval such that a firing rate of the action potential exponentially decays over time during a first time period, and controls the time interval such that the firing rate randomly deviates within a range of a second value less than a first value centered on the first value during a second time period after the first time period, to allow the micro-displacement stimulation element to provide the sense of pressure.

18. The method of claim 15, wherein the signal processor controls the time interval such that the firing rate of the action potential is attenuated or increased over time, and controls an attenuation speed or an increase speed to allow the micro-displacement stimulation element to provide the sense of pressure in which a pressure changes over time.

19. The method of claim 14, wherein the signal processor controls the displacement signal to allow the micro-displacement stimulation element to provide the sense of texture such that a pulse width of the displacement signal has two or more different pulse width of 1 msec or more, or an amplitude of the displacement signal has two or more different magnitudes of 100 um or less, and the waveform of the displacement signal becomes a sine wave.

20. The method of claim 14, wherein the signal processor controls the displacement signal to allow the micro-displacement stimulation element to selectively fire the action potentials of the plurality of tactile receptors such that the displacement signal is a pulse signal and at least one condition of the amplitude of the displacement signal and a time interval between pulses of the displacement signal changes over time.

21. The method of claim 14, wherein the waveform of the displacement signal includes a sine wave and a rectangular wave.

22. The method of claim 14, wherein the signal processor controls the displacement signal to allow the micro-displacement stimulation element to selectively fire the action potentials of the plurality of tactile receptors such that the displacement signal is a sine wave signal and at least one condition of a magnitude of the amplitude and a pulse width of the displacement signal changes over time.

23. The method of claim 14, wherein the micro-displacement stimulation element is an element capable of applying a sine wave vibration having a pulse width of 1 msec or more and an amplitude of 100 um or less to the skin.
